(19) United States
(12) Patent Application Publication
(10) Pub. No.: US 2023/0211127 A1
(43) Pub. Date: Jul. 6, 2023

(54) STEERABLE MEDICAL DEVICE, HANDLE FOR A MEDICAL DEVICE, AND METHOD FOR OPERATING A MEDICAL DEVICE

(71) Applicant: Boston Scientific Medical Device Limited, Galway (IE)

(72) Inventors: Doran Avivi, Toronto (CA); Jan-Hung Chen, Georgetown (CA)

(21) Appl. No.: 18/181,979

(22) Filed: Mar. 10, 2023

Related U.S. Application Data
(63) Continuation of application No. PCT/IB2021/057570, filed on Aug. 17, 2021.
(60) Provisional application No. 63/076,472, filed on Sep. 10, 2020.

(51) Int. Cl. A61M 25/01

(52) U.S. Cl. CPC A61M 25/0136; A61M 25/0147; A61B 2017/00318

(57) ABSTRACT

A handle for a medical device includes a handle body and a knob that is rotatable with respect to the handle body. A first gear is associated with the knob. A slide assembly is housed within the handle body or the knob. The slide assembly includes a first lead screw. A second gear is associated with the slide assembly. A third gear is housed within the handle body or the knob and is coupled between the first gear and the second gear. Rotation of the knob drives rotation of the first gear, rotation of the first gear drives rotation of the third gear, rotation of the third gear drives rotation of the second gear, and rotation of the second gear drives rotation of the lead screw.

### CROSS-REFERENCE TO RELATED APPLICATIONS

This application is a continuation of and claims the benefit of International Application Number PCT/IB2021/057570, entitled “STEERABLE MEDICAL DEVICE, HANDLE FOR A MEDICAL DEVICE, AND METHOD FOR OPERATING A MEDICAL DEVICE,” and filed Aug. 17, 2021, which claims the benefit of U.S. Provisional Application Number 63/076,472, entitled STEERABLE MEDICAL DEVICE, HANDLE FOR A MEDICAL DEVICE, AND METHOD FOR OPERATING A MEDICAL DEVICE,” and filed Sep. 10, 2020, which are hereby incorporated by reference in their entireties.

### FIELD

This document relates to medical devices. More specifically, this document relates to steerable medical devices such as steerable sheaths, catheters, and introducers.

### SUMMARY

The following summary is intended to introduce the reader to various aspects of the detailed description, but not to define or delimit any invention.

Steerable medical devices are disclosed. According to some aspects, a steerable medical device includes a handle having a handle body and a knob that is rotatable with respect to the handle body. A first gear is associated with the knob. A slide assembly is housed within the handle. The slide assembly includes a first lead screw. A second gear is associated with the slide assembly. A third gear is housed within the handle and is coupled between the first gear and the second gear. Rotation of the knob drives rotation of the first gear, rotation of the first gear drives rotation of the third gear, rotation of the third gear drives rotation of the second gear, and rotation of the second gear drives rotation of the lead screw. An elongate tool extends from the handle, and least one control wire is coupled between the slide assembly and the tool. Rotation of the lead screw causes tensioning of the control wire, and tensioning of the control wire causes deflection of the tool.

In some examples, the first gear is an internal gear of the rotatable knob, the second gear is a sun gear that is fixed to the lead screw, and the third gear is a planetary gear that is coupled between the internal gear and the sun gear. The medical device can optionally further include an additional planetary gear coupled between the internal gear and the sun gear. The planetary gear(s) can optionally be fixed in position with respect to the internal gear and the sun gear.

In some examples, the slide assembly further includes a slider coupled to the lead screw and to the control wire. Rotation of the lead screw can cause translation of the slider with respect to the lead screw, and translation of the slider can cause tensioning of the control wire.

In some examples, the first gear is a first bevel gear that is mounted to the knob, the second gear is a second bevel gear that is mounted to the slide assembly, and the third gear is a third bevel gear coupled between the first bevel gear and second bevel gear. The first bevel gear can optionally be fixed to the knob. The first bevel gear, second bevel gear, third bevel gear, and lead screw can optionally be translatable with respect to the knob and the handle body.

In some examples, rotation of the lead screw causes translation of the lead screw, and translation of the lead screw causes tensioning of the control wire.

In some examples, the tool is a sheath, a catheter, or an introducer.

Handles for medical devices are also disclosed. According to some aspects, a handle for a medical device includes a handle body and a knob that is rotatable with respect to the handle body. A first gear is associated with the knob. A slide assembly is housed within the handle body or the knob, and the slide assembly includes a first lead screw. A second gear is associated with the slide assembly. A third gear is housed within the handle body or the knob and is coupled between the first gear and the second gear. Rotation of the knob drives rotation of the first gear, rotation of the first gear drives rotation of the third gear, rotation of the third gear drives rotation of the second gear, and rotation of the second gear drives rotation of the lead screw.

In some examples, the first gear is an internal gear of the rotatable knob, the second gear is a sun gear that is fixed to the lead screw, and the third gear is a planetary gear that is coupled between the internal gear and the sun gear. The handle can optionally further include an additional planetary gear coupled between the internal gear and the sun gear. The planetary gear(s) can optionally be fixed in position with respect to the internal gear and the sun gear.

In some examples, the slide assembly further includes a slider coupled to the lead screw. Rotation of the lead screw can cause translation of the slider with respect to the lead screw.

In some examples, the first gear is a first bevel gear that is mounted to the knob, the second gear is a second bevel gear that is fixed to the slide assembly, and the third gear is a third bevel gear coupled between the first bevel gear and second bevel gear. The first bevel gear can optionally be fixed to the knob. The first bevel gear, second bevel gear, third bevel gear, and lead screw can optionally be translatable with respect to the knob and the handle body.

In some examples, rotation of the lead screw causes translation of the lead screw.

Methods for operating medical devices are also disclosed. According to some aspects, a method for operating a medical device includes: a. rotating a knob of a handle; b. transmitting rotation of the knob to a lead screw of a slide assembly via at least one gear housed within the handle, to cause rotation of the lead screw; c. causing tensioning of a control wire by rotation of the lead screw; and d. causing deflection of a tool by tensioning of the control wire.

In some examples, step a. includes rotating an internal gear of the knob.

In some examples, step b. includes rotating a rotating a planetary gear engaged with the internal gear, and rotating a sun gear that is engaged with the planetary gear and fixed to the lead screw.

In some examples, step c. includes translating a slider along the lead screw to cause tensioning of the control wire.

In some examples, step a. includes rotating a first bevel gear mounted to the knob.

In some examples, step b. includes rotating a third bevel gear engaged with the first bevel gear, and rotating a second bevel gear engaged with the third bevel gear and mounted to the lead screw.

In some examples, step c. includes translating a slider along the lead screw to cause tensioning of the control wire.

In some examples, step c. includes translating the lead screw to cause tensioning of the control wire.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical device;

FIG. 2 is a cutaway plan view of the handle of the medical device of FIG. 1, showing the interior of the handle;

FIG. 3 is a cross-section taken along line 3-3 in FIG. 2;

FIG. 4 is a cutaway plan view of a handle of another medical device, showing the interior of the handle;

FIG. 5 is a cross-section taken along line 5-5 in FIG. 4;

FIG. 6 is a cutaway plan view of a handle of another medical device, showing the interior of the handle;

FIG. 7 is a cutaway plan view of a handle of another medical device, showing the interior of the handle; and

FIG. 8 is a cutaway plan view of a handle of another medical device, showing the interior of the handle.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are steerable medical devices that include a handle and a tool such as a sheath, a catheter, or an introducer. The handle can enable the user to manipulate or steer the tool in a desired direction. More specifically, the handle can include a knob that is rotatably coupled to a body of the handle. In operation, rotation of the knob in a first direction can allow the user to steer or deflect the tool in a first direction, whereas the rotation of the knob in a second direction can allow the user to steer or deflect the tool in a second direction. The rotation of the knob can be converted into a deflection of the tool via a slide assembly, which can be within the handle, and one or more control wires, which are connected between the slide assembly and the tool. The slide assembly can include a lead screw (also referred to as a bolt). Rotation of the knob can cause rotation of the lead screw. Rotation of the lead screw can cause linear translation of the lead screw, or can cause linear translation of a slider along the lead screw. This translation, whether of the lead screw itself or of the slider, causes a tensioning of the control wire(s), which results in a deflection of the tool. For simplicity, details of the slider and the control wires are not disclosed herein. However, related sliders and control wires are disclosed in, for example, U.S. Pat. No. 10,661,057 (Davies et al.), which is incorporated herein by reference in its entirety. Furthermore, steerable medical devices including sliders and control wires are sold Baylis Medical Company, Inc. (Montreal, Canada) under the brand name SureFlex® Steerable Guiding Sheath.

The steerable medical devices disclosed herein are configured to avoid or minimize or reduce unwanted relaxing in the deflection of the tool, and to avoid or minimize or reduce slop in the steering of the tool. More specifically, in the steerable medical devices disclosed herein, a gear train is provided between the knob and the slide assembly. The gear train can allow for the lead screw to have a relatively small thread pitch, which can reduce slop. Furthermore, the gear train can allow for the lead screw to rotate faster than the knob. This can compensate for the reduced linear displacement per knob revolution as a result of the small thread pitch. Furthermore, the gear train can provide mechanical resistance to unwinding, without increasing overall system friction.

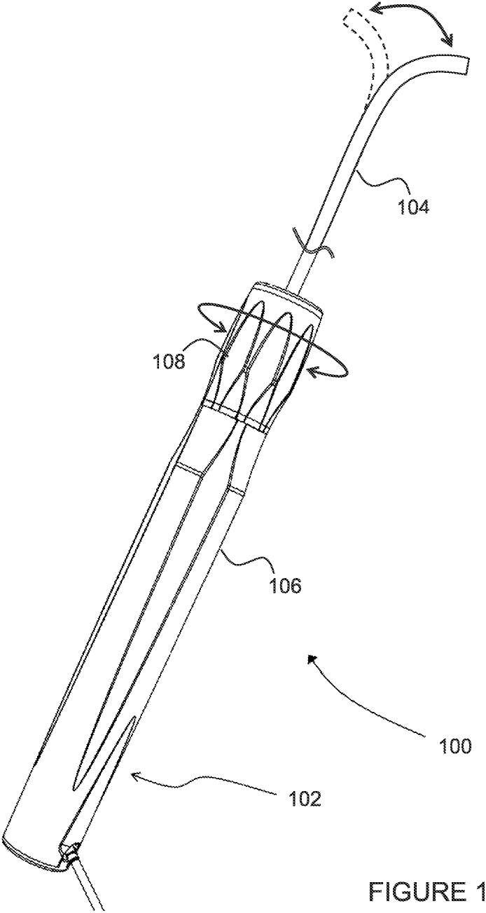

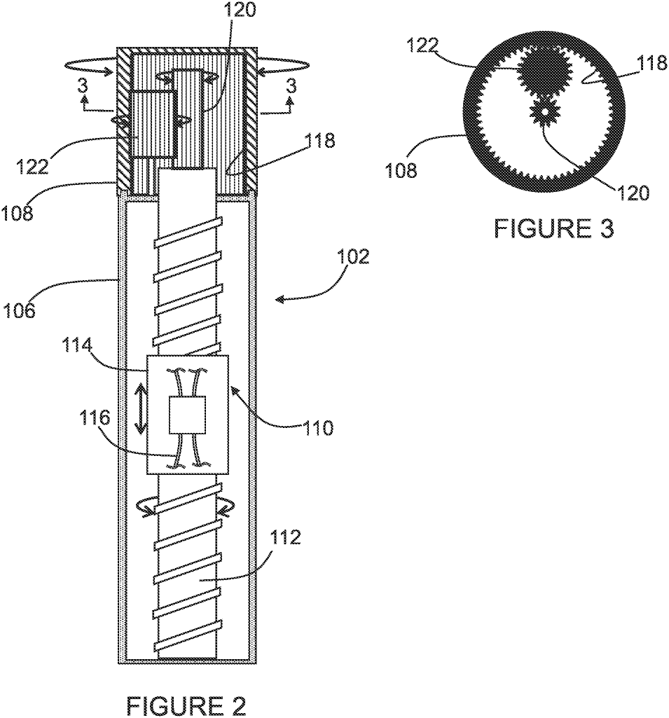

Referring now to FIG. 1, an example steerable medical device 100 is shown. The steerable medical device 100 generally includes a handle 102 and an elongate tool 104 extending from the handle 102. The tool 104 can be, for example (but not limited to), a sheath, a catheter, or an introducer. The handle 102 includes a handle body 106 and a knob 108 that is rotatable with respect to the handle body 106. The knob 108 can be rotated to steer the tool 104. Rotation of the knob 108 in a first direction (e.g. clockwise) can cause the tool 104 to deflect in a first direction (i.e. to the configuration shown in dotted line in FIG. 1), and rotation of the knob 108 in a second direction (e.g. counter-clockwise) can cause the tool 104 to deflect in a second direction (i.e. back to the configuration shown in solid line in FIG. 1). The tool 104 is shown only in FIG. 1, and for simplicity is omitted in FIGS. 2 to 7.

Referring to FIG. 2, in which the handle body 106 and knob 108 are cutaway to show the interior of the handle 102, a slide assembly 110 is housed within the handle 102. As will be described in further detail below, the knob 108 is coupled to the slide assembly 110 to drive movement of the slide assembly 110 by rotation of the knob 108. More specifically, in the example shown, the slide assembly 110 includes a lead screw 112 and a slider 114 coupled to the lead screw. The slider 114 is received on the lead screw 112 and is translatable along the lead screw 112. Rotation of the knob 108 is transmitted to the lead screw 112 to cause rotation of the lead screw 112. Rotation of the lead screw 112 causes translation of the slider 114 along the lead screw 112. A control wire 116 is in turn coupled between the slider 114 of the slide assembly 110 and the tool 104 (shown in FIG. 1). Movement of the slide assembly 110 - i.e. translation of the slider 114 caused by rotation of the lead screw 112 - causes tensioning of the control wire 116, and tensioning of the control wire 116 causes deflection of the tool 104. As mentioned above, details of the slider 114 and control wire 116, and the connection between the slider 114, control wire 116 and tool 104 are disclosed in U.S. Pat. No. 10,661,057 (Davies et al.), and are not repeated herein.

Referring still to FIG. 2 and also to FIG. 3, in order to drive movement of the slide assembly 110 by rotation of the knob 108, the handle 102 includes a gear train, namely a first gear 118 associated with the knob 108, a second gear 120 associated with the slide assembly 110, and a third gear 122 coupled between the first gear 118 and the second gear 120. Rotation of the knob 108 drives rotation of the first gear 118, rotation of the first gear 118 drives rotation of the third gear 122, rotation of the third gear 122 drives rotation of the second gear 120, and rotation of the second gear 120 drives rotation of the lead screw 112.

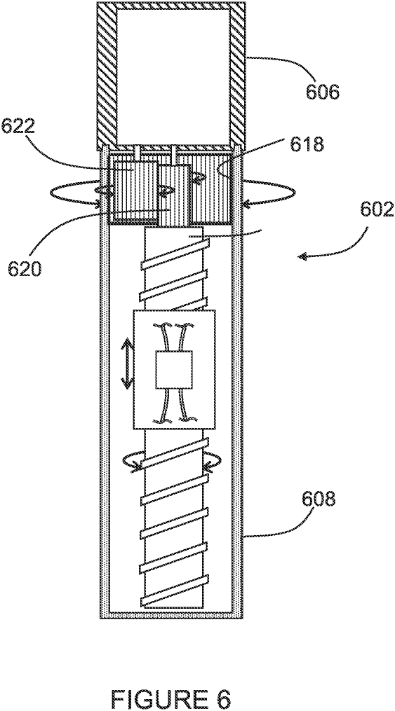

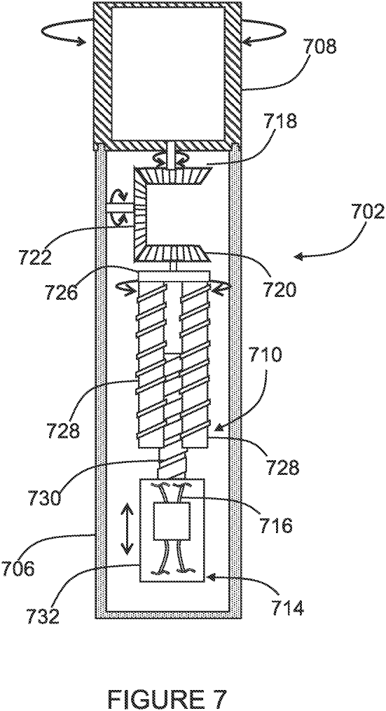

As used herein, the term ‘associated with’ can mean mounted to, secured to, fixed to, adhered to, connected to, integral with, part of, movable with, and/or built into. Accordingly, the statement that “the handle 102 includes a first gear 118 associated with the knob 108” can mean, for example, that the first gear 118 is built into the knob 108 (as will be described below with reference to FIGS. 2 and 3), or that the first gear 118 is a separate piece that is mounted to the knob 108 (as will be described below with reference to FIGS. 6 and 7).

Referring still to FIGS. 2 and 3, in the example shown, the first gear 118 is an internal gear of the knob 108. The second gear 120 is a sun gear that is fixed to the lead screw 112. The third gear 122 is a planetary gear that is mounted to the handle body 106 and is coupled between the internal gear (i.e. the first gear 118) and the sun gear (i.e. the second gear 120). Rotation of the knob 108 causes rotation of the internal gear (i.e. the first gear 118), rotation of the internal gear causes rotation of the planetary gear (i.e. the third gear 122), rotation of the planetary gear causes rotation of the sun gear (i.e. the second gear 120), and rotation of the sun gear causes rotation of the lead screw 112.

In the example shown, the planetary gear (i.e. the third gear 122) is fixed in position with respect to the internal gear (i.e. the first gear 118) and sun gear (i.e. the second gear 120). That is, the planetary gear does not orbit the sun gear. In alternative examples, the planetary gear can orbit the sun gear.

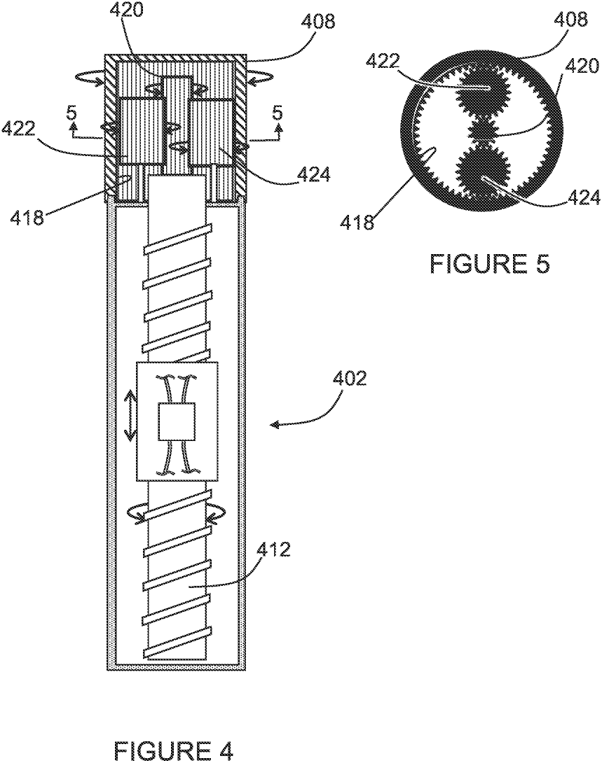

In the example shown in FIGS. 2 and 3, the handle 102 includes one planetary gear. In alternative examples, the handle can include additional planetary gears. One such example is shown in FIGS. 4 and 5. In FIGS. 4 and 5, features that are like those of FIGS. 1 to 3 are identified with like reference numerals, incremented by 300. Furthermore, for brevity, only certain features of FIGS. 4 and 5 are described. In the example shown, the handle 402 includes a first gear 418 in the form of an internal gear of the knob 408, a second gear 420 in the form of a sun gear fixed to the lead screw 412, and a third gear 422 and fourth gear 424 in the form of planetary gears coupled between the first gear 418 and the second gear 420.

In the examples of FIGS. 1 to 3, the rotatable knob 108 is a relatively small part of the handle 102 and is at the top of the handle 120, whereas the handle body 106 is a relatively large part of the handle 102 and is at the bottom of the handle 102. An alternative example is shown in FIG. 6. In FIG. 6, features that are like those of FIGS. 1 to 3 are identified with like reference numerals, incremented by 500. Furthermore, for brevity, only certain features of FIG. 6 are described. In the example of FIG. 6, the rotatable knob 608 is the relatively large part of the handle 602 at the bottom of the handle 602, and the handle body 606 is the relatively small part at the top of the handle 602. In this example, the first gear 618, second gear 620, and third gear 622 are housed within the knob 608.

Referring now to FIG. 7, another example of a handle 702 is shown. In FIG. 7, features that are like those of FIGS. 1 to 3 are identified with like reference numerals, incremented by 600. Furthermore, for brevity, only certain features of FIG. 7 are described. In the handle 702, the first gear 718 is a first bevel gear that is fixed to and rotatable with the knob 708, the second gear 720 is a second bevel gear that is mounted to the slide assembly 710 to drive movement of the slide assembly 710 (as will be described below), and the third gear 722 is a third bevel gear that is mounted to and rotatable with respect to the handle body 706. The third gear 722 is coupled between the first gear 718 and the second gear 720, so that rotation of the knob 708 with respect to the handle body 706 drives rotation of the first gear 718 with respect to the handle body 706, rotation of the first gear drives rotation of the third gear 722 with respect to the handle body 706, and rotation of the third gear 722 drives rotation of the second gear 720 with respect to the handle body 706.

Referring still to FIG. 7, in the example shown, the slide assembly 710 includes a plate 726 that is rotatable with the second gear 720, and a set of four primary lead screws 728 (only two of which are visible) rotatably mounted to and rotatable with the plate 726. The slider 714 includes a secondary lead screw 730 and a slider body 732 fixed to the secondary lead screw 730. The secondary lead screw 730 is positioned between the primary lead screws 728 and is engaged with the primary lead screws 728. Rotation of the plate causes 726 orbiting of the primary lead screws 726 around the secondary lead screw 730, which causes translation of the secondary lead screw 730 and slider body 732. Translation of the slider body 732 causes tensioning of the control wire 716 and deflection of the tool (not shown).

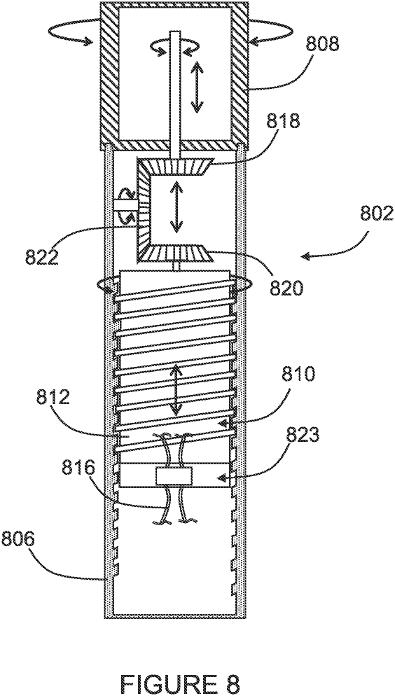

Referring now to FIG. 8, another example of a handle 802 is shown. In FIG. 8, features that are like those of FIGS. 1 to 3 are identified with like reference numerals, incremented by 700. Furthermore, for brevity, only certain features of FIG. 8 are described. In this example, the slide assembly 810 includes a lead screw 812 that itself is translatable with respect to the handle body 806 to cause tensioning of the control wire 816 (i.e. a separate slider is not provided). That is, the slide assembly 810 includes a lead screw 812, and the inside surface of the handle body 806 is threaded and is threadably engaged with the lead screw 812. Rotation of the lead screw 812 with respect to the handle body 806 causes translation of the lead screw 812 along the handle body, and translation of the lead screw 812 causes tensioning of the control wire 816. In this example, the control wires are mounted to a bearing 823 that is fixed to the lead screw 812 in order to prevent twisting of the wires during translation. In alternative examples, the control wires may be routed along the lead screw axis of rotation, in order to avoid or prevent entanglement.

Referring still to FIG. 8, in the example shown, the gears are configured similarly to the example of FIG. 7 - that is, the handle 802 includes a first gear 818 in the form of a first bevel gear that is fixed to and rotatable with the knob 808, a second gear 820 in the form of a second bevel gear that is mounted to the lead screw 812 to drive rotation of the lead screw 812, and a third gear 822 in the form of a third bevel gear that is coupled between the first gear 818 and the second gear 820. However, in the example of FIG. 8, the first gear 818, second gear 820, and third gear 822 are translatable with respect to the handle body 806 and the knob 808, together with the lead screw 812.

In an alternative example, the lead screw 812 of FIG. 8 can be replaced by a set of lead screws (similar to those of FIG. 7),

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be revisited.

We claim:

1. A steerable medical device comprising:
a handle having a handle body and a knob that is rotatable with respect to the handle body;
a first gear associated with the knob;
a slide assembly housed within the handle, wherein the slide assembly comprises a first lead screw;
a second gear associated with the slide assembly;
a third gear housed within the handle and coupled between the first gear and the second gear, wherein rotation of the knob drives rotation of the first gear, rotation of the first gear drives rotation of the third gear, rotation of the third gear drives rotation of the second gear, and rotation of the second gear drives rotation of the lead screw;
an elongate tool extending from the handle; and
at least one control wire coupled between the slide assembly and the tool, whereby rotation of the lead screw causes tensioning of the control wire, and tensioning of the control wire causes deflection of the tool.

2. The steerable medical device of claim 1, wherein
the first gear is an internal gear of the rotatable knob;
the second gear is a sun gear that is fixed to the lead screw; and
the third gear is a planetary gear that is coupled between the internal gear and the sun gear.

3. The steerable medical device of claim 2, further comprising an additional planetary gear coupled between the internal gear and the sun gear.

4. The steerable medical device of claim 2, wherein the planetary gear is fixed in position with respect to the internal gear and the sun gear.

5. The steerable medical device of claim 1, wherein the slide assembly further comprises a slider coupled to the lead screw and to the control wire, wherein rotation of the lead screw causes translation of the slider with respect to the lead screw, and translation of the slider causes tensioning of the control wire.

6. The steerable medical device of claim 1, wherein
the first gear is a first bevel gear that is mounted to the knob;
the second gear is a second bevel gear that is mounted to the slide assembly; and
the third gear is a third bevel gear coupled between the first bevel gear and second bevel gear.

7. The steerable medical device of claim 6, wherein the first bevel gear is fixed to the knob.

8. The steerable medical device of claim 6, wherein
the first bevel gear, second bevel gear, third bevel gear, and lead screw are translatable with respect to the knob and the handle body.

9. The steerable medical device of claim 8, wherein rotation of the lead screw causes translation of the lead screw, and translation of the lead screw causes tensioning of the control wire.

10. The steerable medical device of claim 1, wherein the tool is a sheath, a catheter, or an introducer.

11. A handle for a medical device, the handle comprising:
a handle body and a knob that is rotatable with respect to the handle body;
a first gear associated with the knob;
a slide assembly housed within the handle body or the knob, wherein the slide assembly comprises a first lead screw;
a second gear associated with the slide assembly; and
a third gear housed within the handle body or the knob and coupled between the first gear and the second gear, wherein rotation of the knob drives rotation of the first gear, rotation of the first gear drives rotation of the third gear, rotation of the third gear drives rotation of the second gear, and rotation of the second gear drives rotation of the lead screw.

12. The handle of claim 11, wherein
the first gear is an internal gear of the rotatable knob;
the second gear is a sun gear that is fixed to the lead screw; and
the third gear is a planetary gear that is coupled between the internal gear and the sun gear.

13. The handle of claim 12, further comprising an additional planetary gear coupled between the internal gear and the sun gear.

14. The handle of claim 12, wherein the planetary gear is fixed in position with respect to the internal gear and the sun gear.

15. The handle of claim 11, wherein the slide assembly further comprises a slider coupled to the lead screw, wherein rotation of the lead screw causes translation of the slider with respect to the lead screw.

16. The handle of claim 11, wherein
the first gear is a first bevel gear that is mounted to the knob;
the second gear is a second bevel gear that is fixed to the slide assembly; and
the third gear is a third bevel gear coupled between the first bevel gear and second bevel gear.

17. The handle of claim 16, wherein the first bevel gear is fixed to the knob.

18. The handle of claim 16 wherein
the first bevel gear, second bevel gear, third bevel gear, and lead screw are translatable with respect to the knob and the handle body.

19. The handle of claim 18, wherein rotation of the lead screw causes translation of the lead screw.

20. A method for operating a medical device, comprising:
a. rotating a knob of a handle;
b. transmitting rotation of the knob to a lead screw of a slide assembly via a gear train housed within the handle, to cause rotation of the lead screw;
c. causing tensioning of a control wire by rotation of the lead screw; and
d. causing deflection of a tool by tensioning of the control wire.
